(19) United States
(12) Patent Application Publication
(10) Pub. No.: US 2005/0106688 A1
(43) Pub. Date: May 19, 2005

(54) Method for producing L-amino acid

(76) Inventors: Akira Imaizumi, Kawasaki-shi (JP); Rustem Saidovich Shakulov, Moscow (RU); Andrey Leonidovich Briukhanov, Moscow (RU)

Correspondence Address: AJINOMOTO CORPORATE SERVICES, LLC;INTELLECTUAL PROPERTY DEPARTMENT, 1120 CONNECTICUT AVE., N.W., WASHINGTON, DC, 20036, US

(21) Appl. No.: 10/862,424

(22) Filed: Jun. 8, 2004

(30) Foreign Application Priority Data
Jun. 11, 2003 (JP) 2003-166654
Mar. 26, 2004 (JP) 2004-91708

(51) Int. Cl. C12P 13/04; C12P 13/08; C12N 1/21

(52) U.S. Cl. 435/106; 435/115; 435/252.3

(57) ABSTRACT

An L-amino acid is produced by culturing a bacterium having an ability to produce an L-amino acid in a medium to allow accumulation of the L-amino acid in a culture and by collecting the L-amino acid from the culture, the bacterium being modified so that intracellular ppGpp synthesis ability is increased.

### BACKGROUND OF THE INVENTION

1. Field of the Invention

The present invention relates to the fermentation industry. More specifically, the present invention relates to a bacterium having an ability to produce an L-amino acid and a method for producing an L-amino acid utilizing such a bacterium.

2. Description of the Related Art

To date, it has been widely clarified that ppGpp (guanosine-3′-diphosphate-5′-diphosphate) and pppGpp (guanosine-3′-triphosphate-5′-diphosphate) each play an important role in microbial cell signaling. It is also known that ppGpp is essential for inducing adaptation of microorganisms to survive under conditions whereby intracellular amino acids and sugars necessary for their proliferation have been exhausted.

It has been reported that ppGpp is produced by the RelA protein, the gene product of the relA gene, and by the SpoT protein, a gene product of spoT gene, in Escherichia coli. In addition, the nucleotide and amino acid sequences of these genes and proteins have also been reported (GenBank accession J04039, Metzger, S. et al., J. Biol. Chem., 1988, 263 (30), 15699-15704, GenBank accession AE000442 U00096).

The RelA protein is present in bacterial cells in the form of binding to a ribosome. When a non-aminoacylated tRNA binds to a ribosome, it serves as an amino acid depletion signal that triggers synthesis of pppGpp from GTP and GDP which is catalyzed by the RelA protein on the ribosome. It is also known that the SpoT protein catalyzes the following three kinds of reactions: the reaction from pppGpp to ppGpp, the reaction from GTP to ppGpp, and the reaction from ppGpp to GTP. Hereinafter, both ppGpp and pppGpp are collectively referred to as “ppGpp” because it is believed that their physiological functions in a cell are identical (Cashel, M., Gentry, D. R., Hernadez, V. J., and Vinella, D., The stringent response, In: Neidhardt, F. C. et al. (ed) Escherichia coli and Salmonella; Cellar and Molecular Biology, 2^{nd} edition, 1458-1496 (ASM Press, Washington D.C., 1996). It is known that when ppGpp accumulates in cells via RelA protein activity as a result of sudden depletion of amino acids, the cells generally exhibit a series of responses including termination of ribosome synthesis, degradation of ribosomal proteins, promotion of expression of genes in various amino acid biosynthetic pathways, and so forth. These are generally referred to as stringent responses, and it is widely known that these responses are necessary for the cells to survive under starvation conditions in that they supply depleted amino acids (Cashel, M., Gentry, D. R., Hernadez, V. J., and Vinella, D., The stringent response, In: Neidhardt, F. C. et al. (ed) Escherichia coli and Salmonella; Cellar and Molecular Biology, 2^{nd} edition, 1458-1496 (ASM Press, Washington D.C., 1996).

Analytical experiments conducted to date include improving protein production utilizing a recombinant Escherichia coli by eliminating production of ppGpp (Dedhia, N. et al., Biotechnol. Bioeng., 1997, Vol. 53, 379-386), improving production of antibiotics by modifying the ppGpp-binding sites of ribosomal proteins and RNA polymerase in Actinomyces (Hu, H. and Ochi, K., Appl. Environ. Microbiol., 2001, Vol. 67, 1885-18921, Hu, H., Zhang, Q., and Ochi, K., J. Bacteriol., 2002, Vol. 184, 3984-3991), and so forth.

However, no research has been reported to date concerning the relationship between amino acid biosynthetic systems and the ability to produce ppGpp.

### SUMMARY OF THE INVENTION

An object of the present invention is to improve an ability to produce an L-amino acid of a bacterium and a bacterium having an improved ability to produce an L-amino acid.

It is an object of the present invention to provide a method for producing an L-amino acid comprising culturing a bacterium having an ability to produce an L-amino acid in a medium to allow accumulation of the L-amino acid in a culture, and collecting the L-amino acid from the culture, wherein the bacterium is modified so that an activity to synthesize ppGpp is increased.

It is a further object of the invention to provide the method as described above, wherein said bacterium has been modified so that an activity of an enzyme which synthesizes ppGpp is increased.

It is a further object of the invention to provide the method as described above, wherein said enzyme is selected from the group consisting of a RelA protein and a catalytic domain of the RelA protein.

It is a further object of the invention to provide the method as described above, wherein the activity of said RelA protein is increased by
a) increasing the copy number of a relA gene or a partial region of the relA gene which encodes a catalytic domain of the RelA protein, or
b) modifying an expression regulatory sequence of a relA gene so that intracellular expression of said relA gene or said partial region of said relA gene of the bacterium is enhanced.

It is a further object of the invention to provide the method as described above, wherein said RelA protein is selected from the group consisting of
(A) a protein which has the amino acid sequence of SEQ ID NO: 20; and
(B) a protein which has the amino acid sequence of SEQ ID NO: 20 and includes substitutions, deletions, insertions, or additions of one or several amino acid residues,
and wherein said protein has an activity to synthesize ppGpp.

It is a further object of the invention to provide the method as described above, wherein said RelA protein is encoded by a DNA selected from the group consisting of:
(a) a DNA which has the nucleotide sequence of SEQ ID NO: 19,
(b) a DNA which is hybridizable with the nucleotide sequence of SEQ ID NO: 19 under stringent conditions.

It is a further object of the invention to provide the method as described above, wherein said L-amino acid is selected from the group consisting of L-glutamic acid, L-threonine, L-isoleucine, L-lysine, L-histidine, L-valine, L-arginine, L-leucine, L-phenylalanine and L-tryptophan.

It is a further object of the invention to provide the method as described above, wherein said L-amino acid is selected from the group consisting of L-glutamic acid, L-threonine, L-isoleucine and L-lysine.

It is a further object of the invention to provide the method as described above, wherein said bacterium belongs to the genus Escherichia.

It is an even further object of the invention to provide a bacterium which has an ability to produce an L-amino acid and which has been modified so that an activity of intracellular RelA protein is increased.

It is a further object of the invention to provide the method as described above, wherein said RelA protein has homology of at least 70% to the amino acid sequence in SEQ ID No. 20.

It is a further object of the invention to provide the method as described above, wherein said RelA protein has homology of at least 90% to the amino acid sequence in SEQ ID No. 20.

According to the present invention, L-amino acid production of bacteria can be improved.

### BRIEF DESCRIPTION OF THE DRAWINGS

FIG. 1 shows construction of plasmids pM14 and pM15.

FIG. 2 shows construction of plasmid pMD4041-cat-2Tfd.

FIG. 3 shows construction of plasmids pSTVrelA and pSTVrelA*.

### DESCRIPTION OF THE PREFERRED EMBODIMENTS

The inventors of the present invention assiduously studied in order to achieve the aforementioned objects. As a result, it was found that the ability to produce an L-amino acid can be enhanced by increasing the ability to produce ppGpp, in particular, by increasing the activity of the RelA protein to synthesize ppGpp. Thus, the present invention was accomplished.

Hereafter, the present invention will be explained in detail.

<1> Bacterium of the Present Invention

The bacterium of the present invention is a bacterium which has an ability to produce an L-amino acid and which is modified so that the ability to synthesize ppGpp in the cell is increased.

The bacterium of the present invention is not particularly limited so long as an the bacterium's ability to produce an L-amino acid can be increased by increasing the ability to synthesize ppGpp. Examples of the bacterium include, but are not limited to, bacteria belonging to the genus Escherichia, such as Escherichia coli, coryneform bacteria, such as Brevibacterium lactofermentum, bacteria belonging to the genus Serratia, such as Serratia marcescens, bacteria belonging to the genus Bacillus, such as Bacillus subtilis, and so forth.

The term “ability to produce an L-amino acid” used in the present invention means an ability to cause accumulation of the L-amino acid in a medium when the bacterium of the present invention is cultured in the medium. This ability to produce an L-amino acid may be an inherent property of a wild-type strain of a bacterium or a property imparted or enhanced by breeding.

Examples of the L-amino acid of the present invention include L-glutamic acid, L-threonine, L-isoleucine, L-lysine, L-histidine, L-valine, L-arginine, L-leucine, L-phenylalanine, L-tryptophan and so forth. Of these, L-glutamic acid, L-threonine, L-isoleucine and L-lysine are preferred.

Specific examples of a bacterium having an ability to produce an L-amino acid include, but are not limited to, the following: if L-glutamic acid is desired as the target L-amino acid, the Escherichia coli MG1655ΔsucA (see the examples section), Escherichia coli AJ12624 (FERM BP-3853, see French Patent Laid-open Publication No. 2,680,178) and L-valine resistant strains of Escherichia coli, such as Escherichia coli B11, Escherichia coli K-12 (ATCC10798), Escherichia coli B (ATCC11303) and Escherichia coli W (ATCC9637), Brevibacterium lactofermentum AJ12475 (FERM BP-2922, see U.S. Pat. No. 5,272,067) and so forth can be used;
if L-threonine is desired, Escherichia coli VKPM B-3996 (see U.S. Pat. No. 5,175,107), Escherichia coli MG442 (VKPM B-1628, see Gusyatiner et al., Genetika (in Russian), 14, pp. 947-956, 1978, U.S. Pat. No. 4,278,765), Corynebacterium acetoacidophilum AJ12318 (FERM BP-1172, see U.S. Pat. No. 5,188,949) and so forth can be used;
if L-isoleucine is desired, Escherichia coli KX141 (VKPM B-4781, see European Patent Application Laid-open No. 519,113), Brevibacterium flavum AJ12149 (FERM BP-759, see U.S. Pat. No. 4,656,135) and so forth can be used;
if L-lysine is desired, Escherichia coli AJ11442 (NRRL B-12185, FERM BP-1543, see U.S. Pat. No. 4,346,170), Escherichia coli WC196 strain (FERM BP-5252, WO96/17930), Brevibacterium lactofermentum AJ3990 (ATCC31269, see U.S. Pat. No. 4,066,501) and so forth can be used;
if L-phenylalanine is desired, Escherichia coli AJ 12604 (FERM BP-3579, see European Patent Application Laid-open No. 488,424), Brevibacterium lactofermentum AJ12637 (FERM BP-4160, see French Patent Application Laid-open No. 2,686,898) and so forth can be used;
if L-leucine is desired, strains having β-2-thienylalanine resistance, strains having resistance to β-2-thienylalanine and β-hydroxyleucine (see Japanese Patent Publication (Kokoku) No. 62-34397 for all of the above), strains having resistance to 4-azaleucine resistance or 5,5,5-trifluoroleucine (Japanese Patent Laid-open (Kokai) No. 8-70879), Escherichia coli AJ11478 (FERM P-5274, see Japanese Patent Publication No. 62-34397), Brevibacterium lactofermentum AJ3718 (FERM P-2516, see U.S. Pat. No. 3,970,519) and so forth can be used;
if L-valine is desired, Escherichia coli VL1970 (VKPM B-4411, (see European Patent Application Laid-open No. 519,113), Brevibacterium lactofermentum AJ 12341 (FERM BP-1763, see U.S. Pat. No. 5,188,948) and so forth can be used; and
if L-homoserine is desired, the NZ10 strain, which is a Leu⁺ revertant of the Escherichia coli C600 strain (see Appleyard R. K., Genetics, 39, pp. 440-452, 1954) can be used.

Of the above strains, the Escherichia coli MG1655ΔsucA is obtained by disrupting the sucA gene, which encodes the E1 subunit of ΔKGDH (α-ketoglutarate dehydrogenase) from the MG1655 strain (available from E. coli Genetic Stock Center (Yale University, Dept. Biology, Osborn Memorial Labs., 06511-7444 New Haven, Conn., U.S.A., P.O. Box 6666) (see examples section). The nucleotide sequence and the amino acid sequence encoded thereby are known (see, for example, GenBank accession X00661). Furthermore, disruption of choromosomal sucA gene of Escherichia coli is known (see EP 0 670 370 B1).

Furthermore, the Escherichia coli B-3996 strain is deficient in the thrC gene, utilizes sucrose, and has a leaky mutation in the ilvA gene. This strain has a mutation in the rht gene, which is involved in the high resistance to threonine and homoserine (French Patent Application Laid-open No. 2804971). The B-3996 strain harbors the plasmid pVIC40, which is obtained by inserting a thrA*BC operon containing a mutant thrA gene encoding aspartokinase-homoserine dehydrogenase I, for which feedback inhibition by threonine is substantially desensitized, into a vector derived from RSF1010. The B-3996 strain was deposited at the Russian National Collection of Industrial Microorganisms (VKPM) (Address: Dorozhny proezd. 1, Moscow 113545, Russian Federation) on Apr. 7, 1987 and received an accession number of B-3996.

B-3996/pMWD5 (Japanese Patent Laid-open No. 08-047397, U.S. Pat. No. 5,998,178) is obtained by introducing a plasmid pMWD5 into the B-3996 strain. This plasmid contains the ilvGMEDA operon, whereby the region required for attenuation has been removed (Japanese Patent Laid-open No. 08-047397, WO96/26289).

The bacterium of the present invention can be obtained by modifying a bacterium having an ability to produce an L-amino acid such as those mentioned above, so that the activity to synthesize ppGpp of the bacterium is increased. The bacterium of the present invention can also be obtained by imparting an ability to produce an L-amino acid to a bacterium modified so that the activity to synthesize ppGpp of the bacterium is increased, or enhancing an ability to produce an L-amino acid of such a bacterium.

The expression “modified so that the activity to synthesize ppGpp is increased” means that the activity to synthesize ppGpp per cell is increased when compared with that of a non-modified strain, e.g., a wild-type strain. Examples of such a wild-type strain include, but are not limited to, Escherichia coli MG1655, for Escherichia coli.

The activity to synthesize ppGpp of a bacterium can be increased by modifying the bacterium so that an activity of an enzyme to synthesize ppGpp is increased. Examples of a ppGpp synthesis enzyme include RelA protein and SpoT protein. Of these, the RelA protein is preferred. Furthermore, a bacterium may be modified so that the activities of both of RelA protein and SpoT protein is increased.

The activities of the aforementioned bacterial proteins can be increased by, for example, enhancing the expression of the gene encoding RelA (relA) or gene encoding SpoT (spoT). Enhancement of expression levels of these genes can be achieved by increasing the respective copy numbers of relA or spoT. For example, a gene fragment containing relA or spoT can be ligated to a vector that functions in a bacterium, preferably a multi-copy type vector, to prepare recombinant DNA and then used to transform the bacterium.

The origin of the relA gene or spoT gene is not particularly limited so long as the genes function in the host bacterium to which these genes are introduced. However, a gene derived from the same species as the host or an analogous species is preferred.

The nucleotide sequences of relA and spoT of Escherichia coli are known (relA: GenBank accession AE000362, nucleotide numbers 1667 to 3901, spoT: GenBank accession AE000442 U00096, nucleotide numbers 3791 to 5899). The genes can be obtained by PCR (polymerase chain reaction, see White, T. J. et al., Trends Genet. 5, 185 (1989)) using primers prepared on the basis of the known nucleotide sequences and a chromosomal DNA of a bacterium belonging to the genus Escherichia. relA and spoT homologues of other microorganisms can also be obtained in a similar manner.

The nucleotide sequence of the relA gene and the amino acid sequence of the RelA protein of Escherichia coli are shown in SEQ ID NOS: 19 and 20 respectively. The nucleotide sequence of the spoT gene and the amino acid sequence of the SpoT protein of Escherichia coli are shown in SEQ ID NOS: 21 and 22, respectively.

The genes relA and spoT in the present invention may encode RelA or SpoT including substitution, deletion, insertion, addition or inversion of one or several amino acid residues so long as the activity to synthesize ppGpp of the encoded RelA protein and SpoT protein is not substantially degraded. Although the number of “several” amino acid residues referred to herein differs depending on positions in the three-dimensional structure of the proteins or types of amino acid residues, it may be specifically 2 to 500, preferably 2 to 100, more preferably 2 to 20.

Therefore, changes to RelA or SpoT, such as those described above, are typically conservative changes so as to maintain the activity of RelA or SpoT. Substitution changes include those in which at least one residue in the amino acid sequence has been removed and a different residue inserted in its place. Examples of amino acids which may be substituted for an original amino acid in a RelA or SpoT protein and which are regarded as conservative subsitutions include: Ala substituted with ser or thr; arg substituted with gin, his, or lys; asn substituted with glu, gin, lys, his, asp; asp substituted with asn, glu, or gln; cys substituted with ser or ala; gin substituted with asn, glu, lys, his, asp, or arg; glu substituted with asn, gln, lys, or asp; gly substituted with pro; his substituted with asn, lys, gln, arg, tyr; ile substituted with leu, met, val, phe; leu substituted with ile, met, val, phe; lys substituted with asn, glu, gin, his, arg; met substituted with ile, leu, val, phe; phe substituted with trp, tyr, met, ile, or leu; ser substituted with thr, ala; thr substituted with ser or ala; trp substituted with phe, tyr; tyr substituted with his, phe, or trp; and val substituted with met, ile, leu.

The RelA protein consists of a catalytic domain and a ribosome-binding domain. The ribosome-binding domain of the RelA protein may be deleted in the present invention. In the amino acid sequence of the RelA protein shown in SEQ ID NO: 20, the catalytic domain corresponds to the amino acid numbers 1 to 464. A RelA protein consisting only of the catalytic domain falls within the scope of the RelA protein as described in the present invention. In the present specification, a gene encoding theRelA protein which contains only the catalytic domain may be described as “relA*”.

A DNA encoding a protein substantially identical to RelA or SpoT can be obtained by modifying the nucleotide sequence of the relA or spoT. For example, site-directed mutagenesis can be employed so that substitution, deletion, insertion, addition or inversion of amino acid residues at a specific site of RelA or SpoT. Furthermore, a DNA modified as described above may also be obtained by a conventionally known mutagenesis treatments. The mutagenesis treatment includes a method of treating a DNA before the mutagenesis treatment in vitro with hydroxylamine or the like, and a method of treating a microorganism such as an Escherichia bacterium harboring a DNA before the mutagenesis treatment by ultraviolet irradiation or with a typical mutagenizing agent, such as N-methyl-N′-nitro-N-nitrosoguanidine (NTG) and nitrous acid.

A DNA having a mutation as described above can be expressed in an appropriate cell, and activity of the expression product can be investigated, thereby obtaining a DNA encoding a protein substantially identical to RelA or SpoT. A DNA encoding RelA or SpoT which has a mutation can also be obtained by isolating a DNA that is hybridizable with a probe having a nucleotide sequence comprising, for example, the nucleotide sequence of SEQ ID NO: 19 or 21 or a part thereof, under stringent conditions, and encoding a protein having the activity to synthesize ppGpp from a cell harboring the DNA encoding the mutated RelA or SpoT. The “stringent conditions” referred to herein include conditions under which so-called specific hybrid is formed, and non-specific hybrid is not formed. It is difficult to clearly express this condition using any numerical value. However, for example, the stringent conditions include conditions under which DNAs having high homology, for example, DNAs having homology of not less than 50%, hybridize with each other, but DNAs having homology lower than the above do not hybridize with each other. Alternatively, the stringent conditions are exemplified by a condition whereby DNAs hybridize with each other at a salt concentration corresponding to an ordinary condition of washing in Southern hybridization, i.e., 1×SSC, 0.1% SDS, preferably 0.1×SSC, 0.1% SDS, at 60° C.

A partial sequence of the nucleotide sequence of SEQ ID NO: 19 or 21 can also be used as a probe. Probes can be generated by PCR using oligonucleotides produced on the basis of the nucleotide sequence of SEQ ID NO: 19 or 20 as primers and a DNA fragment containing the nucleotide sequence of SEQ ID NO: 19 or 21 as a template. When a DNA fragment having a length of about 300 bp is used as the probe, the conditions of washing for the hybridization can be, for example, 50° C., 2×SSC and 0.1% SDS.

Specific examples of the DNA encoding a protein substantially identical to RelA include DNA encoding a protein that has homology of preferably 70% or more, more preferably 80% or more, still more preferably 90% or more, particularly preferably 95% or more, with respect to the amino acid sequence shown in SEQ ID NO: 20 and has an activity similar to that of RelA. When the RelA protein consists only of the catalytic domain, it is preferable that the catalytic domain should have a homology to the aforementioned degree. Specific examples of the DNA encoding a protein substantially identical to SpoT include DNA encoding a protein that has homology of preferably 70% or more, more preferably 80% or more, still more preferably 90% or more, particularly preferably 95% or more, with respect to the amino acid sequence shown in SEQ ID NO: 22 and has an activity similar to that of SpoT.

A chromosomal DNA useful as a material for isolating RelA or SpoT can be prepared from a bacterium, which is a DNA donor, by the method of, for example, Saito and Miura (see H. Saito and K. Miura, Biochem. Biophys. Acta, 72, 619 (1963), Text for Bioengineering Experiments, Edited by the Society for Bioscience and Bioengineering, Japan, pp. 97-98, Baifukan, 1992), or the like.

Examples of a primer for relA amplification include relA5 and relA6, which are described in Table 1; and examples of a primer for relA* amplification include relA5 and relA7. Furthermore, examples of a primer for spoT amplification include spoT1 and spoT4.

If a DNA fragment containing relA or spot which is amplified by PCR, is ligated to a vector DNA, which is autonomously replicable in Escherichia coli or the like, in order to prepare a recombinant DNA, subsequent procedures become easier. Examples of the vector autonomously replicable in Escherichia coli include pUC19, pUC18, pHSG299, pHSG399, pHSG398, RSF1010, pBR322, pACYC184, pMW219, pSTV29 and so forth.

In order to prepare a recombinant DNA by ligating relA or spoT and a vector, the vector can be digested with a restriction enzyme corresponding to the terminus of the genes, and ligated using a ligase such as T4 DNA ligase.

In order to introduce the recombinant DNA prepared as described above into a bacterium, any known or previously reported transformation methods can be employed. For instance, specifically into a coryneform bacterium, such methods may include a method of treating recipient cells with calcium chloride so as to increase the permeability of DNA, which has been reported for Escherichia coli K-12 (Mandel, M. and Higa, A., J. Mol. Biol., 53, 159 (1970)), or a method of preparing competent cells from cells which are at the growth phase followed by introducing the DNA thereinto, which has been reported for Bacillus subtilis (Duncan, C. H., Wilson, G. A. and Young, F. E., Gene, 1, 153 (1977)). Furthermore, methods of transformation may include a method of making DNA-recipient cells into protoplasts or spheroplasts, which can easily take up a recombinant DNA, followed by introduction of the recombinant DNA into the cells. This method is known to be applicable to Bacillus subtilis, actinomycetes and yeasts (Chang, S. and Choen, S. N., Molec. Gen. Genet., 168, 111 (1979); Bibb, M. J., Ward, J. M. and Hopwood, O. A., Nature, 274, 398 (1978); Hinnen, A., Hicks, J. B. and Fink, G. R., Proc. Natl. Sci., USA, 75, 1929 (1978)). The transformation can also be performed by the electric pulse method (Japanese Patent Laid-open No. 2-207791).

Increasing the copy number of a gene can also be accomplished by introducing multiple copies of the gene into a chromosomal DNA of a bacterium. Multiple copies of the gene may be introduced into the chromosomal DNA of a bacterium by homologous recombination. This can be performed by targeting a sequence present on the chromosomal DNA in multiple copy number. A repetitive DNA or inverted repeats present at the end of a transposable element can be used as the sequences present on chromosomal DNA in multiple copy number. Alternatively, as disclosed in Japanese Patent Laid-open No. 2-109985, multiple copies of the desired gene can be introduced into chromosomal DNA by incorporating them into a transposon and transferring it.

Besides the above gene amplification methods, RelA or SpoT activity can be enhanced by replacing an expression control sequence, such as promoters of relA or spoT on a chromosomal DNA or plasmid, with stronger control sequences. Examples of strong promoters include lac promoter, trp promoter, trc promoter and so forth. Furthermore, as disclosed in International Patent Publication WO00/18935, by introducing a substitution of several nucleotides into the promoter region of the gene, the promoter can be modified so as to become stronger. Substitution or modification of these promoters enhances expression of the relA or spoT gene, and thus activities of RelA and/or SpoT are enhanced. Methods of modifying expression control sequences may be combined with methods of increasing the copy number of genes.

Substitution of an expression control sequence can be performed, for example, in the same manner as the gene substitution using a temperature-sensitive plasmid, described later. Examples of the temperature-sensitive plasmid of a bacterium belonging to the genus Escherichia include pMAN031 (Yasueda, H. et al, Appl. Microbiol. Biotechnol., 36, 211 (1991)), pMAN997 (WO 99/03988), pEL3 (K. A. Armstrong et. al., J. Mol. Biol. (1984) 175, 331-347) and so forth. pMAN997 is obtained by exchanging the VspI-HindIII fragments of pMAN031 (J. Bacteriol., 162, 1196 (1985)) and pUC19 (Takara Shuzo). These plasmids can autonomously replicate at least at a temperature of 30° C., but cannot autonomously replicate at a temperature of 42° C., in Escherichia coli.

<2> The Method for Producing an L-Amino Acid According to the Present Invention

An L-amino acid can be produced by culturing the bacterium of the present invention obtained as described above in a medium to produce and cause accumulation of an L-amino acid in culture, and collecting the L-amino acid from the culture.

The medium used in the present invention may be a conventionally used well-known medium selected based on type of the bacterium to be utilized or the target L-amino acid. That is, the medium may be a typical medium containing a carbon source, nitrogen source, inorganic ions, as well as other organic components, if necessary. Any special medium is not required for practicing the present invention.

Sugars such as glucose, lactose, galactose, fructose or starch hydrolysate; alcohols such as glycerol or sorbitol; organic acids such as fumaric acid, citric acid or succinic acid and so forth can be used as the carbon source.

Inorganic ammonium salts such as ammonium sulfate, ammonium chloride or ammonium phosphate, organic nitrogen such as soybean hydrolysate, ammonia gas, aqueous ammonia and so forth can be used as the nitrogen source.

It is desirable to allow in the medium required substances such as vitamin B₁, L-homoserine and L-tyrosine or yeast extract in appropriate amounts as organic trace nutrients. Other than the above, potassium phosphate, magnesium sulfate, iron ion, manganese ion and so forth may be added in small amounts, if necessary.

The culture can be performed under conventionally used well-known conditions selected based upon the utilized strain. For example, the culture is preferably performed under aerobic conditions for between 16 and 120 hours. The culture temperature is preferably controlled to be between 25° C. and 45° C., and pH is preferably controlled at between 5 and 8 during the culture. Inorganic or organic, acidic or alkaline substances as well as ammonia gas or the like can be used for pH adjustment.

For collection of the L-amino acid from the medium after completion of the culture, special methods are not required. That is, collection of the target L-amino acid can be performed using a combination of conventionally well-known ion exchange techniques, precipitation techniques, and other techniques depending on the type of the target L-amino acid.

### EXAMPLES

Hereinafter, the present invention will be explained more specifically with reference to the following non-limiting examples.

The amino acids referred to in the following examples are L-amino acids. The primers for PCR used in the following examples are shown in Table 1.

TABLE 1
Sequences of primers

| | | |
|---|---|---|
| Primer | SEQ ID NO: | Sequence |

| | | |
|---|---|---|
| sucA1 | 1 | GCGAATTCCTGCCCCTGACACTAAGACA |
| SucA2 | 2 | CGAGGTAACGTTCAAGACCT |
| SucA3 | 3 | AGGTCTTGAACGTTACCTCGATCCATAACGGGCAGGGCGC |
| SucA4 | 4 | GCGAATTCCCACTTTGTCAGTTTCGATT |
| RelA1 | 5 | GCGAATTCTTGAACTGGTACAGGCAACC |
| RelA2 | 6 | TGTTTAAGTTTAGTGGATGGGTGCGTCTGTTGCAGACAATAC |
| RelA3 | 7 | CCCATCCACTAAACTTAAACATAGCGACACCAAACAGCAAC |
| RelA4 | 8 | GCGAATTCAAGCACTTCACTACTGTTTTC |
| RelA5 | 9 | TTTAAGCTTGCGCGACTGGCGATGC |
| RelA6 | 10 | TTTTCTAGATCCGCACCGCCGGTG |
| RelA7 | 11 | TTTTCTAGATAATTTCAATCTGGTCGCC |
| RelA8 | 12 | GCGAATTCTACGCACTGGCTCAATAATT |
| RelA9 | 13 | GCAAGCTTTGTGACGTTTTATCACGAAA |
| RelA10 | 14 | GCGAATTCTAGATAATTTCAATCTGGTCGCC |
| SpoT1 | 15 | GCGAATTCCGCGGAGTATCTTTATTTTAC |
| SpoT2 | 16 | GCGAATTCCGCGGAGTATCTTTATTTTACAA |
| SpoT3 | 17 | TGTTTAAGTTTAGTGGATGGGACCCGAAACCGAAATTAA |
| SpoT4 | 18 | GCGAATTCTAAAGAATGAGGGCTGAGGC |

### Example 1

<1> Acquisition of a Glutamic Acid-Overproducing Strain of Escherichia coli

First, a sucA gene-disrupted strain of Escherichia coli wild-type strain was constructed in order to obtain a glutamic acid-overproducing strain of Escherichia coli. A deletion-type gene used for gene disruption was prepared by crossover PCR (see Link, A. J., Phillips, D., Church, G. M., J. Bacteriol., 179. pp. 6228-6237, 1997). As primers, sucA1 to sucA4 were used. The sucA1 and sucA4 primers are useful for amplifying the full-length sucA gene, and including about 1000 bp of the flanking regions at either end. The sucA2 and sucA3 primer set is useful for deleting an internal partial sequence of the ORF of the sucA gene.

First, PCR was performed using combinations of the primers sucA1 and sucA2 and the primers sucA3 and sucA4, and using genomic DNA from Escherichia coli wild-type strain MG1655 as a template, prepared by a usual method. In this PCR, the primers sucA1 and sucA2, and the primers sucA4 and sucA3, were used in a molar ratio of 10:1. Secondly, PCR was performed using the resulting product of the first PCR as a template and the sucA1 and sucA4 primers. The sucA gene amplified by this second PCR contained a deletion of an internal sequence of ORF. Both ends of the amplified DNA fragment were digested with the restriction enzyme EcoRI.

The plasmid pMAN997, which has a temperature-sensitive replication origin, was also digested with EcoRI, then purified and ligated to the aforementioned amplified fragment using DNA ligation Kit Ver. 2 (Takara Shuzo). Escherichia coli JM109 competent cells (Takara Shuzo) were transformed with the above ligation reaction mixture, inoculated on an LB agar plate containing 25 μg/ml of ampicillin (“Ap”, Sigma) (LB+Ap plate) and cultured at 30° C. to select colonies. The colonies were cultured in the LB medium containing 25 μg/ml of Ap in test tubes at 30° C., and plasmids were extracted from the cells using Wizard Plus Miniprep (Promega). These plasmids were digested with EcoRI, and a plasmid containing a fragment of the target length was selected as a plasmid for gene disruption (pMANΔsucA).

A target host was transformed with pMANΔsucA, and colonies were selected on LB+Ap plates at 30° C. The selected colonies were cultured overnight at 30° C. in a liquid culture, diluted 10³ times and inoculated on LB+Ap plates, and colonies were selected at 42° C. The selected colonies were spread on LB+Ap plates and cultured at 30° C. Then, the cells corresponding to ⅛ of each plate were suspended in 2 ml of LB medium and cultured at 42° C. for 4 to 5 hours with shaking. The culture broth diluted 10⁵ times was inoculated on an LB plate. Among the resulting colonies, several hundreds of colonies were inoculated on an LB plate and an LB+Ap plate, and their growth was checked to confirm Ap sensitivity or resistance. Colony PCR was performed for several Ap sensitive strains to confirm disruption of the sucA gene. Thus, MG1655ΔsucA was obtained.

<2> Acquisition of a relA Gene-Disrupted Strain and a spoT Gene-Disrupted Strain from a Glutamic Acid-Overproducing Strain of Escherichia coli

From MG1655ΔsucA obtained in <1>, strains were constructed in which the relA gene, the spoT gene, or both were disrupted. Disruption of each gene was performed by crossover PCR. For the relA gene and spoT gene, relA1 to relA4 and spoT1 to spoT4 were used as primers, respectively. The relA1 and relA4 primers, and the spoT1 and spoT4 primers are useful for amplifying the full length relA and spoT genes, respectively, and including about 1000 bp of the flanking regions at either end of these genes. The relA2 and relA3 primer set, and the spoT2 and spoT3 primer set, are useful for deleting internal partial sequences of the ORF of the genes, respectively.

First, PCR was performed using combinations of the primers relA1 and relA2 and the primers relA3 and relA4, and using genomic DNA of the Escherichia coli wild-type strain MG1655 as a template, prepared by a usual method. In this PCR, the primers relA1 and relA2, and the primers relA4 and relA3, were used in a molar ratio of 10:1. Secondly, PCR was performed using the resulting product of the first PCR as a template and the relA1 and relA4 primers.

Furthermore, the first PCR was also performed using combinations of the primers spoT1 and spoT2 and the primers spoT3 and spoT4, and using genomic DNA of the Escherichia coli wild-type strain MG1655 as a template, prepared by a usual method. In this PCR, the primers spoT1 and spoT2, and the primers spoT4 and spoT3, were used in a molar ratio of 10:1. Secondly, PCR was performed using the resulting product of the first PCR as a template and the spoT1 and spoT4 primers.

The relA gene and spoT gene amplified by the second PCR each had a deletion of an internal sequence of ORF.

The plasmid pMAN997 having a temperature sensitive-replication origin was digested with EcoRI, then purified and ligated to the aforementioned amplified fragment using DNA ligation Kit Ver. 2 (Takara Shuzo). Escherichia coli JM109 competent cells (Takara Shuzo) were transformed with the above ligation reaction mixture, inoculated on an LB agar plate containing 25 μg/ml of ampicillin (Ap, Sigma) (LB+Ap plate) and cultured at 30° C. to select colonies. The colonies were cultured in LB medium containing 25 μg/ml of Ap in test tubes at 30° C., and plasmids were extracted from the cells using Wizard Plus Miniprep (Promega). These plasmids were digested with EcoRI, and plasmids containing a fragment of a target length were selected as plasmids for gene disruption (pMANΔrelA and pMANΔspoA).

MG1655ΔsucA obtained in <1> was transformed with pMANΔrelA or pMANΔspoA, and colonies were selected on LB+Ap plates at 30° C. The selected colonies were cultured overnight at 30° C. as liquid culture, diluted 10³ times and inoculated on LB+Ap plates, and colonies were selected at 42° C. The selected colonies were spread on LB+Ap plates and cultured at 30° C. Then, the cells corresponding to ⅛ of each plate were suspended in 2 ml of LB medium and cultured at 42° C. for 4 to 5 hours with shaking. The culture broth diluted 10⁵ times was inoculated on an LB plate. Among the resulting colonies, several hundreds of colonies were inoculated on an LB plate and an LB+Ap plate, and their growth was checked to confirm Ap susceptibility or resistance. Colony PCR was performed for several Ap susceptible strains to confirm disruption of the sucA gene. Thus, MG1655ΔsucAΔrelA and MG1655ΔsucAΔspoT were obtained.

Furthermore, the spoT gene of MG1655ΔsucAΔrelA was disrupted in the same manner as described above to obtain MG1655ΔsucAΔrelAΔspoT.

Each gene-disrupted strain obtained as described above was evaluated for the glutamic acid-producing ability. The strains were cultured in a medium containing 40 g/L of glucose, 1 g/L of MgSO₄.7H₂O, 1 g/L of KH₂PO₄, 16 g/L of (NH₄)₂SO₄, 10 mg/L of FeSO₄.7H₂O, 10 mg/L of MnSO₄.4-5H₂O, 2 g/L of yeast extract and 50 g/L of CaCO₃ contained in a 500-mL volume Sakaguchi flask. The volume of the culture broth at the start of the culture was 20 mL, and the culture was performed at 37° C. for 24 hours with shaking by reciprocal movement at a rotation rate of 120 rpm. The medium, vessels and so forth were all subjected to autoclave sterilization before use. The cell density, glucose concentration and amount of glutamic acid which accumulated in the culture broth were measured. The cell density was determined by measuring turbidity at 562 nm of the culture broth diluted with 0.1 N hydrochloric acid to a suitable concentration using a spectrophotometer (Beckman). The residual glucose concentration and glutamic acid concentration were measured using Biotech Analyzer (Sakura Seiki) for the culture supernatant diluted with water to a suitable concentration after removal of the cells by centrifugation. The results are shown in Table 2.

TABLE 2
Glutamic acid-producing ability of various glutamic
acid-producing bacteria

| | | MG1655 | MG1655 | MG1655 |
|---|---|---|---|---|
| | MG1655 | ΔsucA | ΔsucAΔrelA | ΔsucA |
| Strains | ΔsucA | ΔrelA | ΔspoT | ΔspoT |
| OD₅₆₂ | 16.6 | 10.8 | 8.6 | 15.7 |
| Glucose (g/L) | 0.0 | 20.2 | 22.6 | 0.0 |
| Glutamic acid(g/L) | 13.8 | 3.6 | 3.2 | 15.7 |
| Yield | 34.5% | 18.1% | 18.4% | 39.3% |

As shown in Table 2, it was confirmed that all of the growth, sugar consumption and glutamic acid yield were markedly reduced in the relA-disrupted strains. On the other hand, any other effects were not observed due to the deficiency of the spoT gene.

### Example 2

### Construction of ppGpp Overproducing Plasmid

A strain which overproduces ppGpp was constructed by amplifying the entire relA gene region, or a region encoding the catalytic domain of the relA gene product (relA*). As primers for amplification, four kinds of different plasmids (pMrelA, pMrelA*, pSTVrelA, pSTVrelA*) were constructed.

<1> Construction of pMrelA and pMrelA*

Plasmids pMrelA and pMrelA* were constructed as follows.

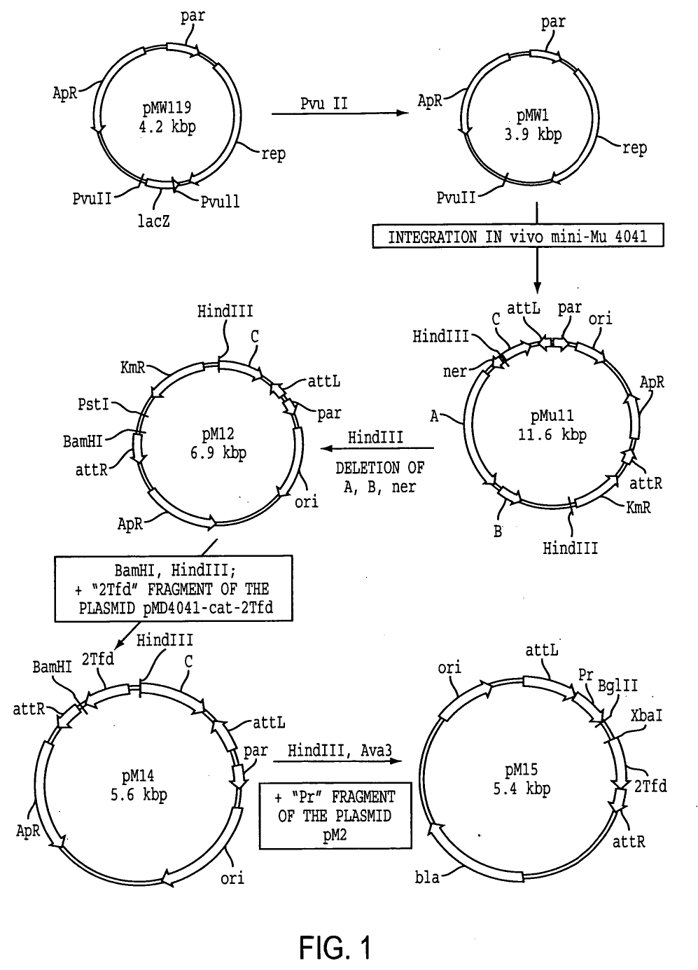

Plasmid pMW119 (Nippon Gene) was digested with the restriction enzyme PvuII and self-cyclized to obtain the plasmid pMW1. Then, the mini-Mud 4041 vector (Miller, “A short course in bacterial genetics”, Cold Springs Harbor Press (1992) 385-400) was incorporated into the plasmid pMW1 in a conventional manner to obtain a plasmid pMu11. pMu11 was digested with the restriction enzyme HindIII and then self-circulated to obtain plasmid pM12, from which the genes A and B encoding the transposase derived from Mu phage and the ner gene encoding a negative control factor were removed. Then, pM12 was digested with restriction enzymes BamHI and HindIII and ligated to a region containing the ter and fd regions (2Tfd), which were excised from the plasmid pMD4041-cat-2Tfd by digestion with the restriction enzymes BamHI and HindIII, to obtain the plasmid pM14 (see FIG. 1).

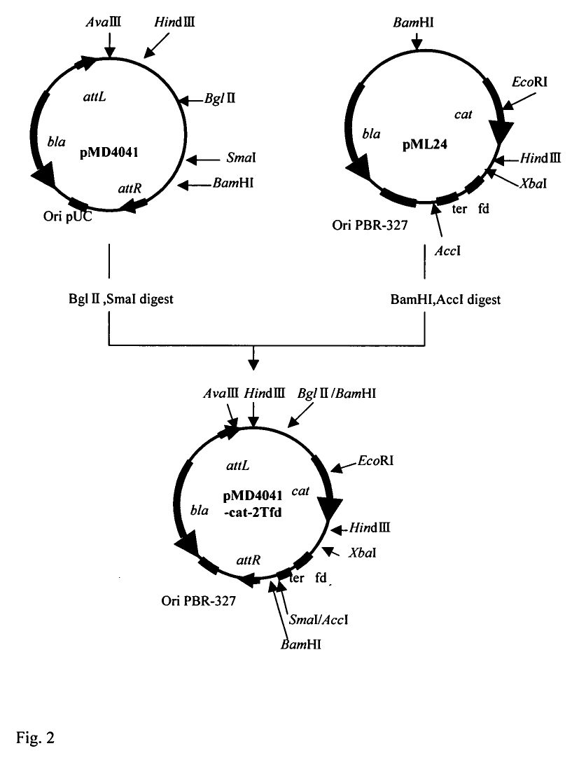

The aforementioned plasmid pMD4041-cat-2Tfd was obtained as follows. The plasmid pML24 (Trukhan et al., Biotechnologiya (in Russian) 4, No. 3 (1988), 325-334; European Patent Application Laid-open No. 1234883) was digested with restriction enzymes BamHI and AccI and blunt-ended with T4 DNA polymerase. This fragment was then ligated with plasmid pMD4041, which had been digested with BglII and SmaI and blunt-ended with DNA polymerase, resulting in plasmid pMD4041-cat-2Tfd (see FIG. 2). The pMD4041 plasmid was obtained by digesting pMu4041 (mini-Mud 4041, Faelen, M., Useful Mu and mini-Mu derivatives, In: Phage Mu, Symonds et al., eds., Cold Spring Harbor Laboratory, New York, 1987, pp. 309-316) with HindIII to excise the A and B genes which encode the transposase of Mu phage, and the ner gene which encodes a negative control factor, and re-cyclizing it (European Patent Application Laid-open No. 1149911).

After digestion with restriction enzymes AvaIII and HindIII, the plasmid pM14 was ligated to a fragment which had been excised from plasmid pM2 (Japanese Patent Laid-open No. 2001-346578, European Patent Application Laid-open No. 1149911) by digestion with restriction enzymes AvaIII and BglII, and containing the P_{R} promoter derived from λ phage. Thus, a plasmid pM15 was obtained (see FIG. 1).

The plasmid pM15 was digested with restriction enzymes HindIII and XbaI to obtain a vector fragment. Furthermore, PCR was performed using genomic DNA from Escherichia coli wild-type strain MG1655 as a template, and relA5 and relA6 as primer. The resulting amplification product was digested with HindIII and XbaI to obtain a DNA fragment containing the relA gene. This DNA fragment and the aforementioned vector fragment (pM15) were ligated using DNA Ligation Kit Ver. 2 (Takara Shuzo). Thus, a plasmid pMrelA was obtained.

Separately, PCR was performed using genomic DNA from MG1655 as a template, and relA5 and relA7 as primers. The amplification product was digested with HindIII and XbaI to obtain a DNA fragment containing the relA* gene. This DNA fragment and the aforementioned vector fragment (pM15) were ligated using DNA Ligation Kit Ver. 2 (Takara Shuzo). Thus, a plasmid pMrelA* was obtained.

<2> Construction of pSTVrelA and pSTVrelA*

Plasmids pSTVrelA and pSTVrelA* were constructed as follows.

The plasmid pSTV29 (Takara Shuzo) was digested with restriction enzymes EcoRI and HindIII to obtain a vector fragment. PCR was performed using genomic DNA from Escherichia coli wild-type strain MG1655 and the relA8 and relA9 primers. The amplification product was digested with restriction enzymes EcoRI and HindIII to obtain a DNA fragment containing the relA gene. These DNA fragments were ligated to obtain a plasmid pSTVrelA.

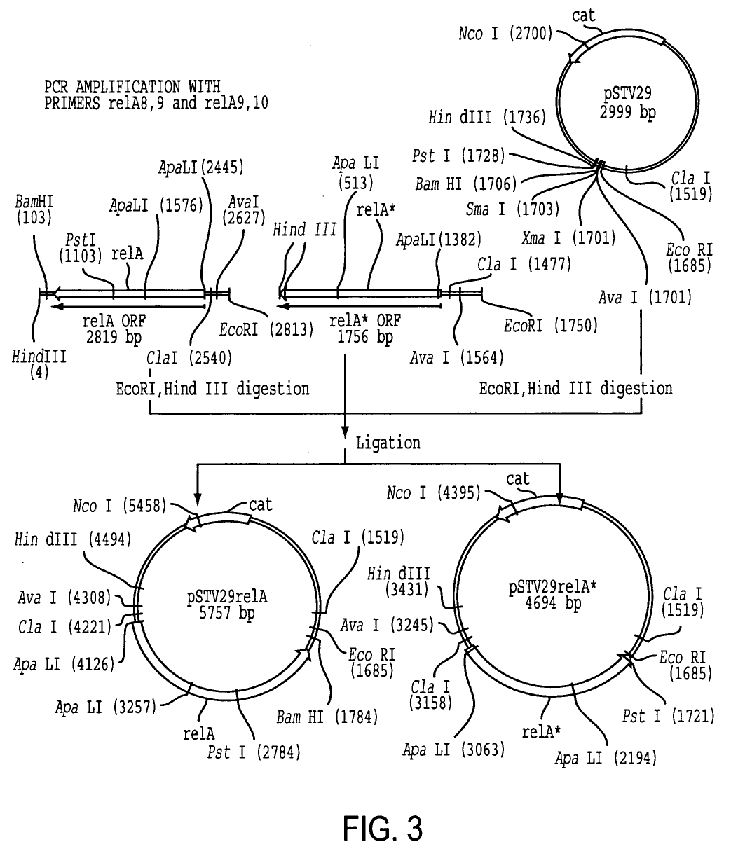

Separately, PCR was performed using genomic DNA from MG1655 as a template, and relA9 and relA10 as primers. The amplification product was digested with EcoRI and HindIII to obtain a DNA fragment containing the relA* gene. This DNA fragment and the aforementioned vector fragment (pSTV29) were ligated to obtain a plasmid pSTVrelA* (see FIG. 3).

### Example 3

### The Effect of the Introduction of the relA Gene into a Glutamic Acid-Overproducing Strain of Escherichia coli and the relA Gene-Disrupted Strain Thereof on Glutamic Acid Production

The plasmids pM15, pMrelA and pMrelA* obtained in Example 2 were used to transform MG1655ΔsucA and MG1655ΔsucAΔrelA obtained in Example 1.

Each transformant was evaluated for the glutamic acid producing ability. The strains were cultured in a medium containing 40 g/L of glucose, 1 g/L of MgSO₄.7H₂O, 1 g/L of KH₂PO₄, 16 g/L of (NH₄)₂SO₄, 10 mg/L of FeSO₄.7H₂O, 10 mg/L of MnSO₄.4-5H₂O, 2 g/L of yeast extract, 50 g/L of CaCO₃ and 100 μg/mL of ampicillin contained in a 500-mL volume Sakaguchi flask. The volume of the culture broth at the start of the culture was 20 mL, and the culture was performed at 37° C. for 24 hours with shaking by reciprocal movement at a rotation rate of 120 rpm. The medium, vessels and so forth were all subjected to autoclave sterilization before use. The cell density, glucose concentration and amount of glutamic acid which accumulated in the culture broth were measured. The cell density was determined by measuring turbidity at 600 nm of the culture broth diluted with 0.1 N hydrochloric acid to a suitable concentration using a spectrophotometer (Beckman). The residual glucose concentration and glutamic acid concentration were measured using Biotech Analyzer (Sakura Seiki) for the culture supernatant diluted with water to a suitable concentration after removal of the cells by centrifugation. The results are shown in Table 3.

TABLE 3
Glutamic acid-producing ability of various glutamic acid-producing bacteria

| | MG1655 | MG1655 | MG1655 | MG1655 | MG1655 | MG1655 |
|---|---|---|---|---|---|---|
| | ΔsucA/ | ΔsucA/ | ΔsucA/ | ΔsucAΔrelA/ | ΔsucAΔrelA/ | ΔsucAΔrelA/ |
| Strain | pM15 | pMrelA* | pMrelA | pM15 | pMrelA* | pMrelA |
| OD₅₆₂ | 17.8 | 17.9 | 17.5 | 7.3 | 7.2 | 16.9 |
| Glucose (g/L) | 0.0 | 0.0 | 0.0 | 21.3 | 23.2 | 0.0 |
| Glutamic acid (g/L) | 15.8 | 16.7 | 19.2 | 1.7 | 2.7 | 18.5 |
| Yield | 39.5% | 41.7% | 48.1% | 9.1% | 16.1% | 47.7% |

In the pMrelA*-introduced strains, improvement of the glutamic acid yield was observed, whereas the effect on recovery of growth of the relA-disrupted strain was not substantially observed. On the other hand, in the pMrelA-introduced strain, growth of the relA-deficient strain completely recovered, and showed improvement in the glutamic acid yield, and the glutamic acid yield was markedly improved compared to control (MG1655ΔsucA/pM15). That is, it was confirmed that the glutamic acid yield was improved due to existence of multiple copies of the relA gene.

### Example 4

### The Effect of the Introduction of the relA and relA* Genes into a Threonine-Overproducing Strain of Escherichia coli on Threonine Production

The plasmids pM15, pMrelA and pMrelA* obtained in Example 2 were used to transform the Escherichia coli threonine-producing strain VKPM B-3996 (Japanese Patent No. 2775948).

Each strain was cultured in a medium containing 40 g/L of glucose, 1 g/L of MgSO₄.7H₂O, 1 g/L of KH₂PO₄, 16 g/L of (NH₄)₂SO₄, 10 mg/L of FeSO₄.7H₂O, 10 mg/L of MnSO₄.4-5H₂O, 2 g/L of yeast extract, 50 g/L of CaCO₃ and 100 μg/mL of ampicillin contained in a 500-mL volume Sakaguchi flask. The volume of the culture broth at the start of the culture was 20 mL, and the culture was performed at 37° C. for 24 hours with shaking by reciprocal movement at a rotation rate of 120 rpm. The medium, vessels and so forth were all subjected to autoclave sterilization before use. The cell density and glucose concentration in the culture broth were measured. The cell density was determined by measuring turbidity at 600 nm of the culture broth diluted with 0.1 N hydrochloric acid to a suitable concentration using a spectrophotometer (Beckman). The residual glucose concentration was measured using Biotech Analyzer (Sakura Seiki) for the culture supernatant diluted with water to a suitable concentration after removal of the cells by centrifugation. The threonine concentration was measured using an amino acid analyzer L-8500 (Hitachi) for the culture supernatant diluted with 0.02 N hydrochloric acid to a suitable concentration after removal of the cells by centrifugation. The results are shown in Table 4.

TABLE 4
Threonine-producing ability of various threonine-producing bacteria

| | | Threonine | Glucose | |
|---|---|---|---|---|
| | | accumulation | concentration | Threonine |
| Strain | OD₅₆₂ | (g/L) | (g/L) | yield |
| B-3996/pM15 | 15.47 | 10.99 | 0.0 | 25.62% |
| B-3996/pMrelA | 16.21 | 12.17 | 0.0 | 29.60% |
| B-3996/pMrelA* | 16.21 | 11.28 | 0.0 | 26.29% |

About 1% of improvement of the threonine yield was observed for the pMrelA*-introduced strain, whereas about 4% of improvement of the threonine yield was observed for the pMrelA-introduced strain. That is, it was confirmed that the threonine yield was improved because of the existence of multiple copies of the relA* or relA genes.

### Example 5

### The Effect of the Introduction of the relA and relA* Genes into an Isoleucine-Overproducing Strain of Escherichia coli on Isoleucine Production

The plasmids pSTV29, pSTVrelA and pSTVrelA* obtained in Example 2 were used to transform the Escherichia coli isoleucine-producing strain B-3996/pMWD5 (Japanese Patent Laid-Open No. 08-047397, U.S. Pat. No. 5,998,178).

Each strain was cultured in a medium containing 40 g/L of glucose, 1 g/L of MgSO₄.7H₂O, 1 g/L of KH₂PO₄, 16 g/L of (NH₄)₂SO₄, 10 mg/L of FeSO₄.7H₂O, 10 mg/L of MnSO₄.4-5H₂O, 2 g/L of yeast extract, 50 g/L of CaCO₃, 100 μg/mL of ampicillin and 25 μg/mL of chloramphenicol contained in a 500-mL volume Sakaguchi flask. The volume of the culture broth at the start of the culture was 20 mL, and the culture was performed at 37° C. for 24 hours with shaking by reciprocal movement at a rotation rate of 120 rpm. The medium, vessels and so forth were all subjected to autoclave sterilization before use. The cell density and glucose concentration in the culture broth were measured. The cell density was determined by measuring turbidity at 600 nm of the culture broth diluted with 0.1 N hydrochloric acid to a suitable concentration using a spectrophotometer (Beckman). The residual glucose concentration was measured using Biotech Analyzer (Sakura Seiki) for the culture supernatant diluted with water to a suitable concentration after removal of the cells by centrifugation. The isoleucine concentration was measured using an amino acid analyzer L-8500 (Hitachi) for the culture supernatant diluted with 0.02 N hydrochloric acid to a suitable concentration after removal of the cells by centrifugation. The results are shown in Table 5.

TABLE 5
Isoleucine-producing ability of various isoleucine-producing bacteria

| | | | Isoleucine | | Glucose | | |
|---|---|---|---|---|---|---|---|
| | OD₅₆₂ | | accumulation (g/L) | | concentration (g/L) | | Isoleucine |
| Strain | 24 hours | 31 hours | 24 hours | 31 hours | 24 hours | 31 hours | yield |
| B-3996/pMWD5, | 15.65 | 14.41 | 6.42 | 8.16 | 4.3 | 0.3 | 19.2% |
| pSTV29 | | | | | | | |
| B-3996/pMWD5, | 11.16 | 14.61 | 3.79 | 8.93 | 25.1 | 1.0 | 21.3% |
| pSTVrelA | | | | | | | |
| B-3996/pMWD5, | 17.97 | 15.77 | 9.23 | 9.46 | 0.7 | 0.4 | 22.3% |
| PSTVrelA* | | | | | | | |

About a 3% improvement of the isoleucine yield was observed for the pSTVrelA*-introduced strain, whereas about a 2% improvement of the isoleucine yield was observed for the pSTVrelA-introduced strain. That is, it was confirmed that the isoleucine yield was improved because of the existence of multiple copies of the relA* gene or relA gene.

### Example 6

### The Effect of the Introduction of the relA and relA* Genes into a Lysine-Overproducing Strain of Escherichia coli on Lysine Production

As an L-lysine-producing strain of Escherichia coli, the WC196 strain was used. This strain was bred by imparting AEC resistance to a W3110 strain which was derived from Escherichia coli K-12. This strain was designated as Escherichia coli AJ13069, and deposited at the National Institute of Bioscience and Human-Technology, Agency of Industrial Science and Technology (presently, the independent administrative agency, International Patent Organism Depositary, National Institute of Advanced Industrial Science and Technology, postal code: 305-8566, Chuo Dai-6,1-1 Higashi 1-Chome, Tsukuba-shi, Ibaraki-ken, Japan) on Dec. 6, 1994 and received an accession number of FERM P-14690. Then, the deposit was converted to an international deposit under the provisions of the Budapest Treaty on Sep. 29, 1995, and received a new accession number of FERM BP-5252 (see International Patent Publication WO96/17930). Furthermore, a pCAB1 plasmid was used to produce a lysine-overproducing strain. This plasmid carried 1) a mutant lysC gene which encodes an aspartokinase for which feedback inhibition by L-lysine was desensitized, 2) a mutant dapA gene which encodes a dihydrodipicolinate synthase for which feedback inhibition by L-lysine was desensitized, and 3) a dapB gene which encodes dihydrodipicolinate reductase (Japanese Patent Laid-open No. 11-192088, U.S. Pat. No. 6,040,160). The Escherichia coli lysine-producing strain WC196 was transformed with this plasmid to obtain the lysine-overproducing strain WC196/pCAB1.

Furthermore, plasmids pM15 and pMrelA obtained in Example 2 were used to transform the Escherichia coli lysine-producing strain WC196/pCAB1 and thereby obtain WC196/pCAB1/pM15 and WC196/pCAB1/pMrelA.

Each strain was cultured in a medium containing 40 g/L of glucose, 1 g/L of MgSO₄.7H₂O, 1 g/L of KH₂PO₄, 16 g/L of (NH₄)₂SO₄, 10 mg/L of FeSO₄.7H₂O, 10 mg/L of MnSO₄.4-5H₂O, 2 g/L of yeast extract, 50 g/L of CaCO₃, 100 μg/mL of ampicillin and 100 μg/mL of streptomycin contained in a 500-mL volume Sakaguchi flask. The volume of the culture broth at the start of the culture was 20 mL, and the culture was performed at 37° C. for 42 hours with shaking by reciprocal movement at a rotation rate of 120 rpm. The medium, vessels and so forth were all subjected to autoclave sterilization before use. The cell density and glucose concentration in the culture broth were measured. The cell density was determined by measuring turbidity at 600 nm of the culture broth diluted with 0.1 N hydrochloric acid to a suitable concentration using a spectrophotometer (Beckman). The residual glucose concentration was measured using Biotech Analyzer (Sakura Seiki) for the culture supernatant diluted with water to a suitable concentration after removal of the cells by centrifugation. The lysine concentration was measured using Biotech Analyzer (Sakura Seiki) for the culture supernatant diluted with water to a suitable concentration after removal of the cells by centrifugation. The results obtained when all of the glucose in the medium was consumed (culture time: 42 hours) are shown in Table 6.

TABLE 6
Lysine-producing ability of various lysine-producing bacteria (results
obtained after 42 hours of culture)

| | | Lysine | |
|---|---|---|---|
| | | accumulation | |
| Strain | OD₅₆₂ | (g/L) | Lysine yield |
| WC196/pCAB1/pM15 | 12.49 (0.037) | 14.7 (1.10) | 36.69% (2.74) |
| WC196/pCAB1/pMrelA | 14.39 (1.66) | 15.8 (0.17) | 39.56% (0.44) |

The numerical values in the parentheses represent standard deviations when n is 3.

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety, including the foreign priority document, JP2003-166654.

1. A method for producing an L-amino acid comprising
a) culturing in a medium a bacterium having an ability to produce said L-amino acid;
b) allowing accumulation of said L-amino acid in said medium, and
c) collecting said L-amino acid from said medium
wherein said bacterium has been modified so that synthesis of ppGpp is increased.

2. The method according to claim 1, wherein said bacterium has been modified so that an activity of an enzyme which synthesizes ppGpp is increased.

3. The method according to claim 2, wherein said enzyme is selected from the group consisting of a RelA protein and a catalytic domain of the RelA protein.

4. The method according to claim 3, wherein the activity of said RelA protein is increased by
a) increasing the copy number of a relA gene or a partial region of the relA gene which encodes a catalytic domain of the RelA protein, or
b) modifying an expression regulatory sequence of a relA gene so that intracellular expression of said relA gene or said partial region of said relA gene of the bacterium is enhanced.

5. The method according to claim 3, wherein said RelA protein is selected from the group consisting of:
(A) a protein which has the amino acid sequence of SEQ ID NO: 20; and
(B) a protein which has the amino acid sequence of SEQ ID NO: 20 and includes substitutions, deletions, insertions, or additions of one or several amino acid residues,
and wherein said protein has an activity to synthesize ppGpp.

6. The method according to claim 3, wherein said RelA protein is encoded by a DNA selected from the group consisting of:
(a) a DNA which has the nucleotide sequence of SEQ ID NO: 19,
(b) a DNA which is hybridizable with the nucleotide sequence of SEQ ID NO: 19 under stringent conditions.

7. The method according to claim 1, wherein said L-amino acid is selected from the group consisting of L-glutamic acid, L-threonine, L-isoleucine, L-lysine, L-histidine, L-valine, L-arginine, L-leucine, L-phenylalanine and L-tryptophan.

8. The method according to claim 7, wherein said L-amino acid is selected from the group consisting of L-glutamic acid, L-threonine, L-isoleucine and L-lysine.

9. The method according to claim 7, wherein said bacterium belongs to the genus Escherichia.

10. A bacterium which has an ability to produce an L-amino acid and which has been modified so that an activity of intracellular RelA protein is increased.

11. The method according to claim 5, wherein said RelA protein has homology of at least 70% to the amino acid sequence in SEQ ID No. 20.

12. The method according to claim 11, wherein said RelA protein has homology of at least 90% to the amino acid sequence in SEQ ID No. 20.


SEQUENCE LISTING (22 sequences)

SEQ ID NO: 1 - DNA, 28 bp, artificial
  primer sucA1
gcgaattcctgcccctgacactaagaca

SEQ ID NO: 2 - DNA, 20 bp, artificial
  primer sucA2
cgaggtaacgttcaagacct

SEQ ID NO: 3 - DNA, 40 bp, artificial
  primer sucA3
aggtcttgaacgttacctcgatccataacgggcagggcgc

SEQ ID NO: 4 - DNA, 28 bp, artificial
  primer sucA4
gcgaattcccactttgtcagtttcgatt

SEQ ID NO: 5 - DNA, 28 bp, artificial
  primer relA1
gcgaattcttgaactggtacaggcaacc

SEQ ID NO: 6 - DNA, 42 bp, artificial
  primer relA2
tgtttaagtttagtggatgggtgcgtctgttgcagacaatac

SEQ ID NO: 7 - DNA, 41 bp, artificial
  primer relA3
cccatccactaaacttaaacatagcgacaccaaacagcaac

SEQ ID NO: 8 - DNA, 29 bp, artificial
  primer relA4
gcgaattcaagcacttcactactgttttc

SEQ ID NO: 9 - DNA, 25 bp, artificial
  primer relA5
tttaagcttgcgcgactggcgatgc

SEQ ID NO: 10 - DNA, 24 bp, artificial
  primer relA6
ttttctagatccgcaccgccggtg

SEQ ID NO: 11 - DNA, 28 bp, artificial
  primer relA7
ttttctagataatttcaatctggtcgcc

SEQ ID NO: 12 - DNA, 28 bp, artificial
  primer relA8
gcgaattctacgcactggctcaataatt

SEQ ID NO: 13 - DNA, 28 bp, artificial
  primer relA9
gcaagctttgtgacgttttatcacgaaa

SEQ ID NO: 14 - DNA, 31 bp, artificial
  primer relA10
gcgaattctagataatttcaatctggtcgcc

SEQ ID NO: 15 - DNA, 29 bp, artificial
  primer spoT1
gcgaattccgcggagtatctttattttac

SEQ ID NO: 16 - DNA, 39 bp, artificial
  primer spoT2
cccatccactaaacttaaacagctttcaaacagatacaa

SEQ ID NO: 17 - DNA, 39 bp, artificial
  primer spoT3
tgtttaagtttagtggatgggacccgaaaccgaaattaa

SEQ ID NO: 18 - DNA, 28 bp, artificial
  primer spoT4
gcgaattctaaagaatgagggctgaggc

SEQ ID NO: 19 - DNA, 2235 bp, Escherichia coli
  CDS (1)..(2235)
atggttgcggtaagaagtgcacatatcaataaggctggtgaatttgatccggaaaaatggatcgcaagtctgggtattaccagccagaagtcgtgtgagtgcttagccgaaacctgggcgtattgtctgcaacagacgcaggggcatccggatgccagtctgttattgtggcgtggtgttgagatggtggagatcctctcgacattaagtatggacattgacacgctgcgggcggcgctgcttttccctctggcggatgccaacgtagtcagcgaagatgtgctgcgtgagagcgtcggtaagtcggtcgttaaccttattcacggcgtgcgtgatatggcggcgatccgccagctgaaagcgacgcacactgattctgtttcctccgaacaggtcgataacgttcgccggatgttattggcgatggtcgatgattttcgctgcgtagtcatcaaactggcggagcgtattgctcatctgcgcgaagtaaaagatgcgccggaagatgaacgtgtactggcggcaaaagagtgtaccaacatctacgcaccgctggctaaccgtctcggaatcggacaactgaaatgggaactggaagattactgcttccgttacctccatccaaccgaatacaaacgaattgccaaactgctgcatgaacggcgtctcgaccgcgaacactacatcgaagagttcgttggtcatctgcgcgctgagatgaaagctgaaggcgttaaagcggaagtgtatggtcgtccgaaacacatctacagcatctggcgtaaaatgcagaaaaagaacctcgcctttgatgagctgtttgatgtgcgtgcggtacgtattgtcgccgagcgtttacaggattgctatgccgcactggggatagtgcacactcactatcgccacctgccggatgagtttgacgattacgtcgctaacccgaaaccaaacggttatcagtctattcataccgtggttctggggccgggtggaaaaaccgttgagatccaaatccgcaccaaacagatgcatgaagatgcagagttgggtgttgctgcgcactggaaatataaagagggcgcggctgctggcggcgcacgttcgggacatgaagaccggattgcctggctgcgtaaactgattgcgtggcaggaagagatggctgattccggcgaaatgctcgacgaagtacgtagtcaggtctttgacgaccgggtgtacgtctttacgccgaaaggtgatgtcgttgatttgcctgcgggatcaacgccgctggacttcgcttaccacatccacagtgatgtcggacaccgctgcatcggggcaaaaattggcgggcgcattgtgccgttcacctaccagctgcagatgggcgaccagattgaaattatcacccagaaacagccgaaccccagccgtgactggttaaacccaaacctcggttacgtcacaaccagccgtgggcgttcgaaaattcacgcctggttccgtaaacaggaccgtgacaaaaacattctggctgggcggcaaatccttgacgacgagctggaacatctggggatcagcctgaaagaagcagaaaaacatctgctgccgcgttacaacttcaatgatgtcgacgagttgctggcggcgattggtggcggggatatccgtctcaatcagatggtgaacttcctgcaatcgcaatttaataagccgagtgccgaagagcaggacgccgccgcgctgaagcaacttcagcaaaaaagctacacgccgcaaaaccgcagtaaagataacggtcgcgtggtagtcgaaggtgttggcaacctgatgcaccacatcgcgcgctgctgccagccgattcctggagatgagattgtcggcttcattacccaggggcgcggtatttcagtacaccgcgccgattgcgaacaactggcggaactgcgctcccatgcgccagaacgcattgttgacgcggtatggggtgagagctactccgccggatattcgctggtggtccgcgtggtagctaatgatcgtagtgggttgttacgtgatatcacgaccattctcgccaacgagaaggtgaacgtgcttggcgttgccagccgtagcgacaccaaacagcaactggcgaccatcgacatgaccattgagatttacaacctgcaagtgctggggcgcgtgctgggtaaactcaaccaggtgccggatgttatcgacgcgcgtcggttgcacgggagttag

SEQ ID NO: 20 - protein, 744 aa, Escherichia coli
MVAVRSAHINKAGEFDPEKWIASLGITSQKSCECLAETWAYCLQQTQGHPDASLLLWRGVEMVEILSTLSMDIDTLRAALLFPLADANVVSEDVLRESVGKSVVNLIHGVRDMAAIRQLKATHTDSVSSEQVDNVRRMLLAMVDDFRCVVIKLAERIAHLREVKDAPEDERVLAAKECTNIYAPLANRLGIGQLKWELEDYCFRYLHPTEYKRIAKLLHERRLDREHYIEEFVGHLRAEMKAEGVKAEVYGRPKHIYSIWRKMQKKNLAFDELFDVRAVRIVAERLQDCYAALGIVHTHYRHLPDEFDDYVANPKPNGYQSIHTVVLGPGGKTVEIQIRTKQMHEDAELGVAAHWKYKEGAAAGGARSGHEDRIAWLRKLIAWQEEMADSGEMLDEVRSQVFDDRVYVFTPKGDVVDLPAGSTPLDFAYHIHSDVGHRCIGAKIGGRIVPFTYQLQMGDQIEIITQKQPNPSRDWLNPNLGYVTTSRGRSKIHAWFRKQDRDKNILAGRQILDDELEHLGISLKEAEKHLLPRYNFNDVDELLAAIGGGDIRLNQMVNFLQSQFNKPSAEEQDAAALKQLQQKSYTPQNRSKDNGRVVVEGVGNLMHHIARCCQPIPGDEIVGFITQGRGISVHRADCEQLAELRSHAPERIVDAVWGESYSAGYSLVVRVVANDRSGLLRDITTILANEKVNVLGVASRSDTKQQLATIDMTIEIYNLQVLGRVLGKLNQVPDVIDARRLHGS

SEQ ID NO: 21 - DNA, 2109 bp, Escherichia coli
  CDS (1)..(2109)
ttgtatctgtttgaaagcctgaatcaactgattcaaacctacctgccggaagaccaaatcaagcgtctgcggcaggcgtatctcgttgcacgtgatgctcacgaggggcaaacacgttcaagcggtgaaccctatatcacgcacccggtagcggttgcctgcattctggccgagatgaaactcgactatgaaacgctgatggcggcgctgctgcatgacgtgattgaagatactcccgccacctaccaggatatggaacagctttttggtaaaagcgtcgccgagctggtagagggggtgtcgaaacttgataaactcaagttccgcgataagaaagaggcgcaggccgaaaactttcgcaagatgattatggcgatggtgcaggatatccgcgtcatcctcatcaaacttgccgaccgtacccacaacatgcgcacgctgggctcacttcgcccggacaaacgtcgccgcatcgcccgtgaaactctcgaaatttatagcccgctggcgcaccgtttaggtatccaccacattaaaaccgaactcgaagagctgggttttgaggcgctgtatcccaaccgttatcgcgtaatcaaagaagtggtgaaagccgcgcgcggcaaccgtaaagagatgatccagaagattctttctgaaatcgaagggcgtttgcaggaagcgggaataccgtgccgcgtcagtggtcgcgagaagcatctttattcgatttactgcaaaatggtgctcaaagagcagcgttttcactcgatcatggacatctacgctttccgcgtgatcgtcaatgattctgacacctgttatcgcgtgctgggccagatgcacagcctgtacaagccgcgtccgggccgcgtgaaagactatatcgccattccaaaagcgaacggctatcagtctttgcacacctcgatgatcggcccgcacggtgtgccggttgaggtccagatccgtaccgaagatatggaccagatggcggagatgggtgttgccgcgcactgggcttataaagagcacggcgaaaccagtactaccgcacaaatccgcgcccagcgctggatgcaaagcctgctggagctgcaacagagcgccggtagttcgtttgaatttatcgagagcgttaaatccgatctcttcccggatgagatttacgttttcacaccggaagggcgcattgtcgagctgcctgccggtgcaacgcccgtcgacttcgcttatgcagtgcataccgatatcggtcatgcctgcgtgggcgcacgcgttgaccgccagccttacccgctgtcgcagccgcttaccagcggtcaaaccgttgaaatcattaccgctccgggcgctcgcccgaatgccgcttggctgaactttgtcgttagctcgaaagcgcgcgccaaaattcgtcagttgctgaaaaacctcaagcgtgatgattctgtaagcctgggccgtcgtctgctcaaccatgctttgggtggtagccgtaagctgaatgaaatcccgcaggaaaatattcagcgcgagctggatcgcatgaagctggcaacgcttgacgatctgctggcagaaatcggacttggtaacgcaatgagcgtggtggtcgcgaaaaatctgcaacatggggacgcctccattccaccggcaacccaaagccacggacatctgcccattaaaggtgccgatggcgtgctgatcacctttgcgaaatgctgccgccctattcctggcgacccgattatcgcccacgtcagccccggtaaaggtctggtgatccaccatgaatcctgccgtaatatccgtggctaccagaaagagccagagaagtttatggctgtggaatgggataaagagacggcgcaggagttcatcaccgaaatcaaggtggagatgttcaatcatcagggtgcgctggcaaacctgacggcggcaattaacaccacgacttcgaatattcaaagtttgaatacggaagagaaagatggtcgcgtctacagcgcctttattcgtctgaccgctcgtgaccgtgtgcatctggcgaatatcatgcgcaaaatccgcgtgatgccagacgtgattaaagtcacccgaaaccgaaattaa

SEQ ID NO: 22 - protein, 702 aa, Escherichia coli
LYLFESLNQLIQTYLPEDQIKRLRQAYLVARDAHEGQTRSSGEPYITHPVAVACILAEMKLDYETLMAALLHDVIEDTPATYQDMEQLFGKSVAELVEGVSKLDKLKFRDKKEAQAENFRKMIMAMVQDIRVILIKLADRTHNMRTLGSLRPDKRRRIARETLEIYSPLAHRLGIHHIKTELEELGFEALYPNRYRVIKEVVKAARGNRKEMIQKILSEIEGRLQEAGIPCRVSGREKHLYSIYCKMVLKEQRFHSIMDIYAFRVIVNDSDTCYRVLGQMHSLYKPRPGRVKDYIAIPKANGYQSLHTSMIGPHGVPVEVQIRTEDMDQMAEMGVAAHWAYKEHGETSTTAQIRAQRWMQSLLELQQSAGSSFEFIESVKSDLFPDEIYVFTPEGRIVELPAGATPVDFAYAVHTDIGHACVGARVDRQPYPLSQPLTSGQTVEIITAPGARPNAAWLNFVVSSKARAKIRQLLKNLKRDDSVSLGRRLLNHALGGSRKLNEIPQENIQRELDRMKLATLDDLLAEIGLGNAMSVVVAKNLQHGDASIPPATQSHGHLPIKGADGVLITFAKCCRPIPGDPIIAHVSPGKGLVIHHESCRNIRGYQKEPEKFMAVEWDKETAQEFITEIKVEMFNHQGALANLTAAINTTTSNIQSLNTEEKDGRVYSAFIRLTARDRVHLANIMRKIRVMPDVIKVTRNRN